(19) United States
(12) Patent Application Publication
(10) Pub. No.: US 2025/0352077 A1
(43) Pub. Date: Nov. 20, 2025

(54) BLOOD PRESSURE MEASURING DEVICE CAPABLE OF MEASURING MERIDIANS

(71) Applicant: K-JUMP HEALTH CO., LTD., New Taipei City (TW)

(72) Inventor: Chao-Man TSENG, New Taipei City (TW)

(21) Appl. No.: 18/663,714

(22) Filed: May 14, 2024

(51) Int. Cl. A61B 5/022; A61B 5/0235

(52) U.S. Cl. CPC A61B 5/02233; A61B 5/0235; A61B 2562/0247

(57) ABSTRACT

A blood pressure measuring device capable of measuring meridians comprises an air path, an air inflation and deflation unit, an airbag unit, a signal sensing unit, a pressure sensing unit and a processing unit. The air path comprises an upstream end and a downstream end. The air inflation and deflation unit is connected to the upstream end of the air path and provides a gas. The airbag unit is connected to the downstream end of the air path. An interior of the airbag unit and the air path form a space for filling the gas. The signal sensing unit is attached to a surface of the airbag unit. The pressure sensing unit is coupled to the space. The processing unit is coupled to the signal sensing unit and the pressure sensing unit.

### FIELD OF THE INVENTION

The invention relates to a blood pressure measuring device, and more particularly to a blood pressure measuring device capable of measuring meridians.

### BACKGROUND OF THE INVENTION

The sphygmomanometer is an important medical device in people's lives. It can accurately measure blood pressure, such as systolic pressure and diastolic pressure. However, in the existing technology, the function of the sphygmomanometer is single and has no variability, it cannot convert other data in the human blood flow into effective information that can provide doctors with judging physical conditions. The scope of application is limited to the function of general blood pressure measurement.

Therefore, Taiwan utility model patent No. TW M465140U discloses a sphygmomanometer-type pulse diagnostic instrument that simultaneously measures blood pressure and pulse condition through an array sensor. However, the parameter samplings of blood pressure measurement and pulse condition measurement are not the same. If only a single detector is used to measure blood pressure and determine pulse condition, the measurement data will not be accurate enough.

### SUMMARY OF THE INVENTION

A main object of the invention is to solve the problem of insufficient accuracy in conventional sphygmomanometer-type pulse diagnosis instruments or pulse diagnosis sphygmomanometers.

In order to solve the above problem, the invention provides a blood pressure measuring device capable of measuring meridians, comprising an air path, an air inflation and deflation unit, an airbag unit, a signal sensing unit, a pressure sensing unit and a processing unit. The air path comprises an upstream end and a downstream end. The air inflation and deflation unit is connected to the upstream end of the air path and provides a gas. The airbag unit is connected to the downstream end of the air path. An interior of the airbag unit and the air path form a space for filling the gas. The signal sensing unit is attached to a surface of the airbag unit. The pressure sensing unit is coupled to the space. The processing unit is coupled to the signal sensing unit and the pressure sensing unit. Wherein the blood pressure measuring device is configured to perform one of the following: when the air inflation and deflation unit supplies the gas to the space and an air pressure in the space reaches and maintains a value, the signal sensing unit captures a continuous pulsation signal of a human body and transmits the continuous pulsation signal to the processing unit; and when the airbag unit receives the gas supplied by the air inflation and deflation unit and continuously discharges the gas from the space, the pressure sensing unit continuously detects a change in air pressure in the space, and transmits a waveform signal generated by the change in air pressure to the processing unit.

In order to solve the above problem, the invention further provides a blood pressure measuring device capable of measuring meridians, comprising an air path, an air inflation and deflation unit, an airbag unit, a signal sensing unit, a pressure sensing unit and a processing unit. The air path comprises an upstream end and a downstream end. The air inflation and deflation unit is connected to the upstream end of the air path and provides a gas. The airbag unit is connected to the downstream end of the air path, the airbag unit is divided into a first inflatable part and a second inflatable part through a welding line, wherein the air path and interiors of the first inflatable part and the second inflatable part forming a space for filling the gas. The signal sensing unit is attached to a surface of the airbag unit. The pressure sensing unit is coupled to the space. The processing unit is coupled to the signal sensing unit and the pressure sensing unit. Wherein the blood pressure measuring device is configured to perform one of the following: when the air inflation and deflation unit supplies the gas to the first inflatable part and an air pressure in the space reaches and maintains a value, the signal sensing unit captures a continuous pulsation signal of a human body and transmits the continuous pulsation signal to the processing unit; and when the second inflatable part receives the gas supplied by the air inflation and deflation unit and continuously discharges the gas from the space, the pressure sensing unit continuously detects a change in air pressure in the space, and transmits a waveform signal generated by the change in air pressure to the processing unit.

In order to solve the above problem, the invention further provides a blood pressure measuring device capable of measuring meridians, comprising an air path, an air inflation and deflation unit, a first airbag unit, a second airbag unit, a signal sensing unit, a pressure sensing unit and a processing unit. The air path comprises an upstream end and a downstream end. The air inflation and deflation unit is connected to the upstream end of the air path and provides a gas. The first airbag unit and the second airbag unit are connected to the downstream end of the air path, wherein the air path and interiors of the first airbag unit and the second airbag unit forming a space for filling the gas. The signal sensing unit is attached to a surface of the airbag unit. The pressure sensing unit is coupled to the space. The processing unit is coupled to the signal sensing unit and the pressure sensing unit. The blood pressure measuring device is configured to perform one of the following: when the air inflation and deflation unit supplies the gas to the first airbag unit and an air pressure in the space reaches and maintains a value, the signal sensing unit captures a continuous pulsation signal of a human body and transmits the continuous pulsation signal to the processing unit; and when the second airbag unit receives the gas supplied by the air inflation and deflation unit and continuously discharges the gas from the space, the pressure sensing unit continuously detects a change in air pressure in the space, and transmits a waveform signal generated by the change in air pressure to the processing unit.

### BRIEF DESCRIPTION OF THE DRAWINGS

FIG. 1 is a schematic diagram of a device architecture of the invention.

FIG. 2 is a perspective view of air path connection according to a first embodiment of the invention.

FIG. 3 is a perspective view of air path connection according to a second embodiment of the invention.

FIG. 4 is a perspective view of air path connection according to a third embodiment of the invention.

### DETAILED DESCRIPTION OF THE PREFERRED EMBODIMENTS

The terminology used herein is for the purpose of describing particular embodiments only and does not limit the invention. The singular forms “a” and “the” used in this specification may also include the plural form unless the context dictates otherwise.

Directional terms used herein, such as up, down, left, right, front, back and their derivatives or synonyms, refer to the orientation of elements in the drawings and do not limit the invention unless the context clearly indicates otherwise.

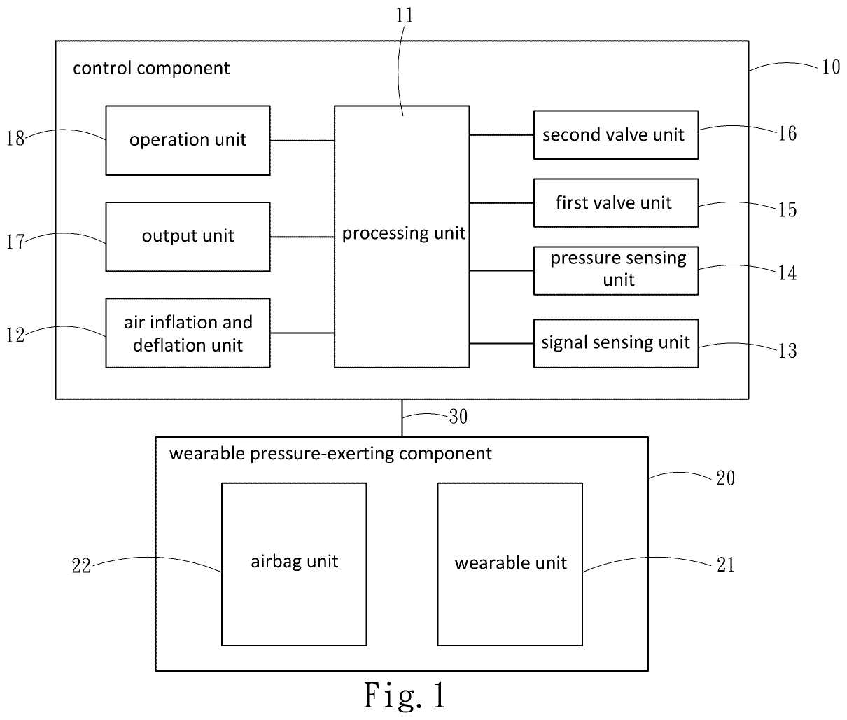

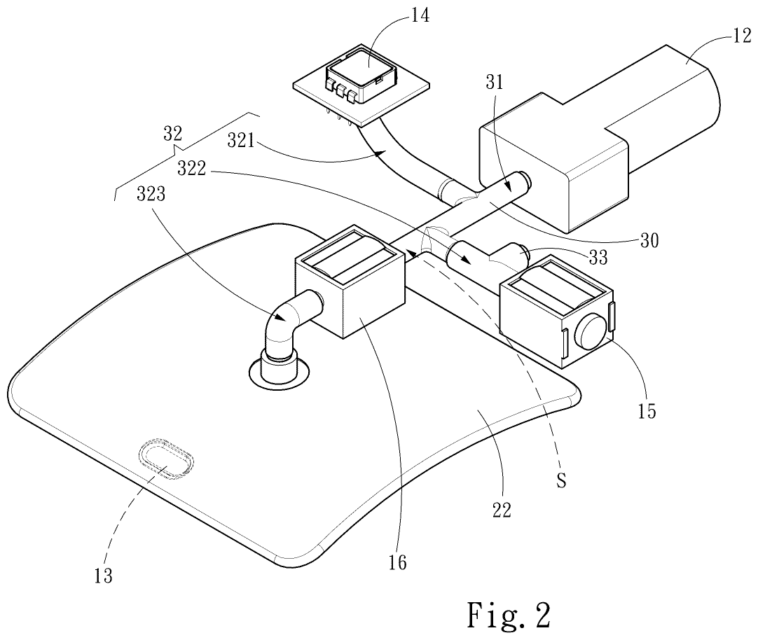

Please refer to FIG. 1 and FIG. 2, the invention provides a blood pressure measuring device 1 capable of measuring meridians. The blood pressure measuring device 1 comprises a control component 10, a wearable pressure-exerting component 20 and an air path 30. The wearable pressure-exerting component 20 is connected to the control component 10 and the control component 10 is fixed on the wearable pressure-exerting component 20 for convenient portability as a whole. The air path 30 communicates with the control component 10 and the wearable pressure-exerting component 20. In this embodiment, the wearable pressure-exerting component 20 is an inflatable wristband, and the control component 10 is a device host for controlling inflation and deflation of the wearable pressure-exerting component 20 and displaying physiological values measured by a user, such as systolic pressure, diastolic pressure, pulse, heart rate, etc.

Please refer to FIG. 1, the control component 10 comprises a processing unit 11, an air inflation and deflation unit 12, a signal sensing unit 13, a pressure sensing unit 14, a first valve unit 15, a second valve unit 16, an output unit 17 and an operation unit 18. The processing unit 11 is coupled to the air inflation and deflation unit 12, the signal sensing unit 13, the pressure sensing unit 14, the first valve unit 15, the second valve unit 16, the output unit 17 and the operation unit 18 to receive or send signals, such as transmitting operation instructions input by the user through the operation unit 18, or receiving pressure changes from the wearable pressure-exerting component 20 through the pressure sensing unit 14.

In one embodiment, the air inflation and deflation unit 12 is a pump. The signal sensing unit 13 is a signal sensor, wherein the signal sensors can be arranged in one group or more than one group, and one group contains one sensor or more than one sensor for measuring a radial artery of the user. The pressure sensing unit 14 is an air pressure sensor. The first valve unit 15 is a relief valve. The second valve unit 16 is a two-way valve. The output unit 17 is a display screen, a speaker or a combination of the above. The operation unit 18 is an operation button and a power switch. In other embodiments, the output unit 17 and the operation unit 18 are integrated into a touch screen for operating and displaying measurement results.

The wearable pressure-exerting component 20 comprises a wearable unit 21 and an airbag unit 22. The wearable unit 21 is disposed on the airbag unit 22 and worn on a wrist of the user to fastened the wearable pressure-exerting component 20 to the user's wrist. The signal sensing unit 13 is disposed on a surface of the airbag unit 22. In one embodiment, the wearable unit 21 can be used to remove and wear the wearable pressure-exerting component 20 repeatedly, such as strap, Velcro, buckle, etc.

Please refer to FIG. 2, in a first embodiment of the invention, the air path 30 is connected to the air inflation and deflation unit 12, the pressure sensing unit 14, the first valve unit 15, the second valve unit 16 and the airbag unit 22.

The air path 30 comprises an upstream end 31, a downstream end 32 and an air leakage part 33. The air inflation and deflation unit 12 is coupled to the upstream end 31 and provides a gas. In one embodiment, the gas is air. The airbag unit 22 is connected to the downstream end 32. An interior of the airbag unit 22 and the air path 30 form a space S for filling the gas. The pressure sensing unit 14, the first valve unit 15, and the second valve unit 16 are respectively connected to the air path 30 and coupled to the space S. The air leakage part 33 is normally open and located at the downstream end 32 for the gas to be discharged from the space S quantitatively. In this embodiment, the pressure sensing unit 14 is disposed at the downstream end 32 to detect a change in air pressure of the space S. The signal sensing unit 13 is disposed on a surface of the airbag unit 22 and is in close contact with the user's skin during use to detect the user's pulse condition. The first valve unit 15 is disposed at the downstream end 32 to enable the air path 30 to communicate externally or close from the outside. The second valve unit 16 is located at an upstream of the airbag unit 22.

In detail, the downstream end 32 of the air path 30 comprises a first downstream end 321, a second downstream end 322 and a third downstream end 323. The pressure sensing unit 14 is connected to the first downstream end 321. The air leakage part 33 and the first valve unit 15 are connected to the second downstream end 322. The airbag unit 22 is connected to the third downstream end 323. The second valve unit 16 is disposed on the air path 30 and is located between the third downstream end 323 and the other downstream ends 32 (the first downstream end 321 and the second downstream end 322). The signal sensing unit 13 is disposed on a surface of the airbag unit 22.

In an example of the first embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 to close and the second valve unit 16 to open, and the gas enters into the airbag unit 22 through the third downstream end 323 to fill the space S with the gas.

When an air pressure in the space S reaches and maintains a value, the second valve unit 16 is closed, the signal sensing unit 13 transmits a sensed continuous pulsation signal (such as amplitude and frequency) of a human body to the processing unit 11, and the pulsation signal is converted into a pulse condition information. Then the processing unit 11 controls the first valve unit 15 and the second valve unit 16 to open to discharge the remaining gas.

The output unit 17 outputs the pulse condition information.

In another example of the first embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 to close and the second valve unit 16 to open, and the gas enters into the airbag unit 22 through the third downstream end 323 to fill the space S with the gas.

The air inflation and deflation unit 12 stops filling the gas. When the airbag unit 22 continuously supplies the gas to the space S, the gas in the space S is quantitatively discharged externally via the air leakage part 33. The pressure sensing unit 14 continuously detects a change in air pressure in the space S and transmits the change in air pressure to the processing unit 11 to obtain a blood pressure information of the user. Then the processing unit 11 controls the first valve unit 15 to open to discharge the remaining gas.

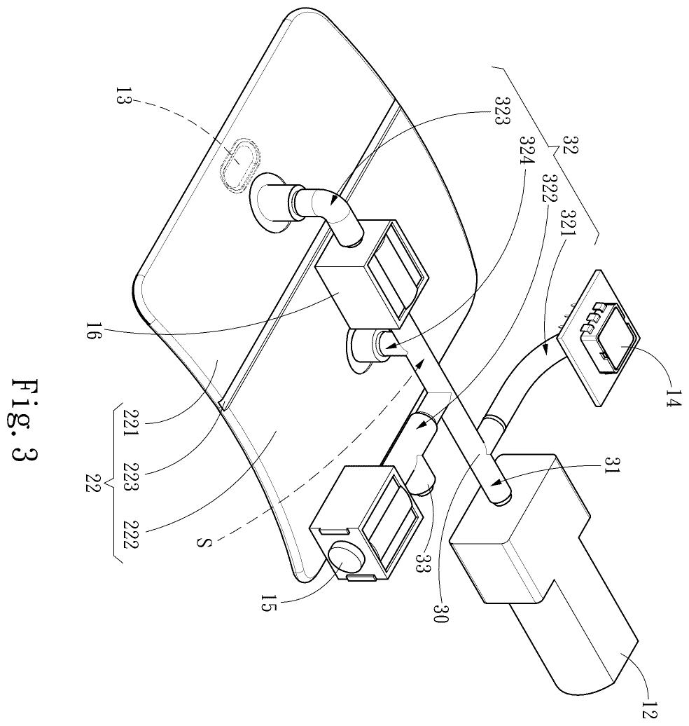

The output unit 17 outputs the blood pressure information. Please refer to FIG. 3, in a second embodiment of the invention, the airbag unit 22 comprises a first inflatable part 221 and a second inflatable part 222. The first inflatable part 221 and the second inflatable part 222 are separated into independent spaces through a welding line 223. The air path 30 and interiors of the first inflatable part 221 and the second inflatable part 222 form the space S for filling the gas. The second valve unit 16 is located at an upstream of the first inflatable part 221, and the downstream end 32 of the air path 30 further comprises a fourth downstream end 324.

In this embodiment, the pressure sensing unit 14 is connected to the first downstream end 321. The air leakage part 33 and the first valve unit 15 are connected to the second downstream end 322. The first inflatable part 221 of the airbag unit 22 is connected to the third downstream end 323. The second inflatable part 222 of the airbag unit 22 is connected to the fourth downstream end 324. The second valve unit 16 is disposed on the air path 30 and located between the third downstream end 323 and the fourth downstream end 324. The signal sensing unit 13 is disposed on a surface of the first inflatable part 221 of the airbag unit 22.

In an example of the second embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 to close and the second valve unit 16 to open, and the gas enters into the first inflatable part 221 and the second inflatable part 222 of the airbag unit 22 through the third downstream end 323 and the fourth downstream end 324 respectively to fill the space S with the gas.

When an air pressure in the space S reaches and maintains a value, the second valve unit 16 is closed, the signal sensing unit 13 transmits a sensed continuous pulsation signal (such as amplitude and frequency) of a human body to the processing unit 11, and the pulsation signal is converted into a pulse condition information. Then the processing unit 11 controls the first valve unit 15 and the second valve unit 16 to open to discharge the remaining gas.

The output unit 17 outputs the pulse condition information.

In another example of the second embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 and the second valve unit 16 to close, and the gas enters into the second inflatable part 222 of the airbag unit 22 through the fourth downstream end 324 to fill the space S with the gas.

The air inflation and deflation unit 12 stops filling the gas. When the second inflatable part 222 continuously supplies the gas to the space S, the gas in the space S is quantitatively discharged externally via the air leakage part 33. The pressure sensing unit 14 continuously detects a change in air pressure in the space S and transmits the change in air pressure to the processing unit 11 to obtain a blood pressure information of the user. Then the processing unit 11 controls the first valve unit 15 to open to discharge the remaining gas.

The output unit 17 outputs the blood pressure information.

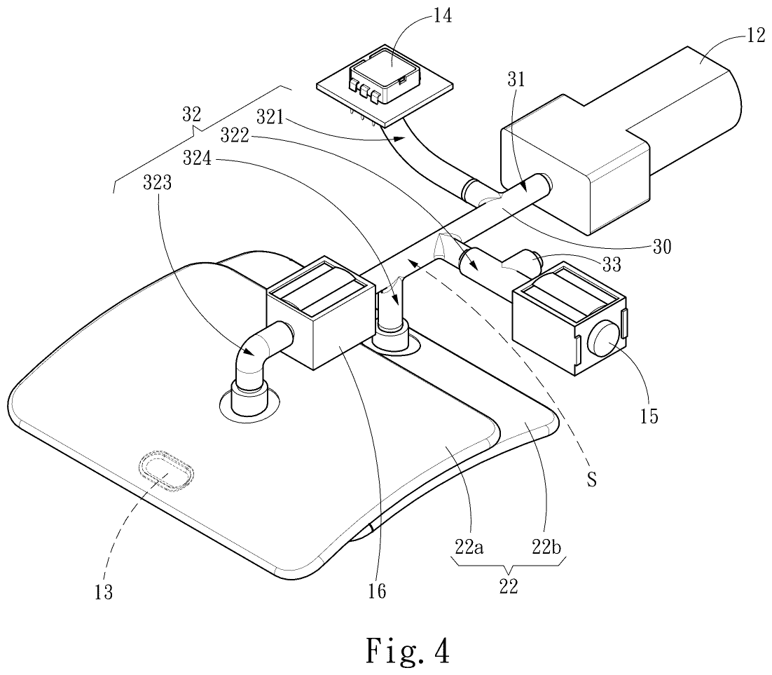

Please refer to FIG. 4, in a third embodiment of the invention, the airbag unit 22 is divided into a first airbag unit 22a and a second airbag unit 22b. The first airbag unit 22a and the second airbag unit 22b are independent spaces. The second valve unit 16 is located at an upstream of the first airbag unit 22a. The air path 30 and interiors of the first airbag unit 22a and the second airbag unit 22b form the space S for filling the gas.

In this embodiment, the pressure sensing unit 14 is connected to the first downstream end 321. The air leakage part 33 and the first valve unit 15 are connected to the second downstream end 322. The first airbag unit 22a is connected to the third downstream end 323. The second airbag unit 22b is connected to the fourth downstream end 324. The second valve unit 16 is disposed on the air path 30 and located between the third downstream end 323 and the fourth downstream end 324. The signal sensing unit 13 is disposed on a surface of the airbag unit 22.

In an example of the third embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 to close and the second valve unit 16 to open, and the gas enters into the first airbag unit 22a and the second airbag unit 22b through the third downstream end 323 and the fourth downstream end 324 respectively to fill the space S with the gas.

When an air pressure in the space S reaches and maintains a value, the second valve unit 16 is closed, the signal sensing unit 13 transmits a sensed continuous pulsation signal (such as amplitude and frequency) of a human body to the processing unit 11, and the pulsation signal is converted into a pulse condition information. Then the processing unit 11 controls the first valve unit 15 and the second valve unit 16 to open to discharge the remaining gas.

The output unit 17 outputs the pulse condition information.

In another example of the third embodiment, the blood pressure measuring device 1 is configured to perform the following.

The wearable pressure-exerting component 20 is worn on the user's wrist, and the blood pressure measuring device 1 is turned on through the operation unit 18.

The air inflation and deflation unit 12 begins to fill the gas, and the gas is input through the upstream end 31 of the air path 30. The processing unit 11 controls the first valve unit 15 and the second valve unit 16 to close, and the gas enters into the second airbag unit 22b through the fourth downstream end 324 to fill the space S with the gas.

The air inflation and deflation unit 12 stops filling the gas. When the second airbag unit 22b continuously supplies the gas to the space S, the gas in the space S is quantitatively discharged externally via the air leakage part 33. The pressure sensing unit 14 continuously detects a change in air pressure in the space S and transmits the change in air pressure to the processing unit 11 to obtain a blood pressure information of the user. Then the processing unit 11 controls the first valve unit 15 to open to discharge the remaining gas.

The output unit 17 outputs the blood pressure information.

Summing up the above, by disposing the signal sensing unit and the pressure sensing unit, the invention enables the blood pressure measuring device to measure the user's pulse condition and blood pressure respectively, and measure the pulse condition and blood pressure respectively through the two sensing units, thereby increasing an accuracy of measurement results. In addition, the user does not need to use a pulse diagnostic instrument and a sphygmomanometer separately for measurement, which has the advantages of reducing equipment purchase costs, shortening measurement time, and saving storage space.

What is claimed is:

1. A blood pressure measuring device capable of measuring meridians, comprising:
an air path, comprising an upstream end and a downstream end;
an air inflation and deflation unit, connected to the upstream end of the air path and providing a gas;
an airbag unit, connected to the downstream end of the air path, an interior of the airbag unit and the air path forming a space for filling the gas;
a signal sensing unit, attached to a surface of the airbag unit;
a pressure sensing unit, coupled to the space; and
a processing unit, coupled to the signal sensing unit and the pressure sensing unit;
wherein the blood pressure measuring device is configured to perform one of the following:
when the air inflation and deflation unit supplies the gas to the space and an air pressure in the space reaches and maintains a value, the signal sensing unit captures a continuous pulsation signal of a human body and transmits the pulsation signal to the processing unit; and
when the airbag unit receives the gas supplied by the air inflation and deflation unit and continuously discharges the gas from the space, the pressure sensing unit continuously detects a change in air pressure in the space, and transmits a waveform signal generated by the change in air pressure to the processing unit.

2. The blood pressure measuring device capable of measuring meridians as claimed in claim 1, wherein the blood pressure measuring device further comprises a first valve unit and a second valve unit, the first valve unit is disposed at the downstream end of the air path to enable the air path to communicate externally or close from the outside, and the second valve unit is disposed on the air path and located at an upstream of the airbag unit.

3. The blood pressure measuring device capable of measuring meridians as claimed in claim 1, wherein the air path further comprises an air leakage part, and the air leakage part is located at the downstream end for the gas to be discharged from the space quantitatively.

4. A blood pressure measuring device capable of measuring meridians, comprising:
an air path, comprising an upstream end and a downstream end;
an air inflation and deflation unit, connected to the upstream end of the air path and providing a gas;
an airbag unit, connected to the downstream end of the air path, the airbag unit divided into a first inflatable part and a second inflatable part through a welding line, wherein the air path and interiors of the first inflatable part and the second inflatable part forming a space for filling the gas;
a signal sensing unit, attached to a surface of the airbag unit;
a pressure sensing unit, coupled to the space; and
a processing unit, coupled to the signal sensing unit and the pressure sensing unit;
wherein the blood pressure measuring device is configured to perform one of the following:
when the air inflation and deflation unit supplies the gas to the first inflatable part and an air pressure in the space reaches and maintains a value, the signal sensing unit captures a continuous pulsation signal of a human body and transmits the pulsation signal to the processing unit; and
when the second inflatable part receives the gas supplied by the air inflation and deflation unit and continuously discharges the gas from the space, the pressure sensing unit continuously detects a change in air pressure in the space, and transmits a waveform signal generated by the change in air pressure to the processing unit.

5. The blood pressure measuring device capable of measuring meridians as claimed in claim 4, wherein the blood pressure measuring device further comprises a first valve unit and a second valve unit, the first valve unit is disposed at the downstream end of the air path to enable the air path to communicate externally or close from the outside, and the second valve unit is disposed on the air path and located between the first inflatable part and the second inflatable part.

6. The blood pressure measuring device capable of measuring meridians as claimed in claim 4, wherein the air path further comprises an air leakage part, the air leakage part is located at the downstream end for the gas to be discharged from the space quantitatively.

7. A blood pressure measuring device capable of measuring meridians, comprising:
an air path, comprising an upstream end and a downstream end;
an air inflation and deflation unit, connected to the upstream end of the air path and providing a gas;
a first airbag unit and a second airbag unit, connected to the downstream end of the air path, wherein the air path and interiors of the first airbag unit and the second airbag unit forming a space for filling the gas;
a signal sensing unit, attached to a surface of the airbag unit;
a pressure sensing unit, coupled to the space; and
a processing unit, coupled to the signal sensing unit and the pressure sensing unit;
wherein the blood pressure measuring device is configured to perform one of the following:
when the air inflation and deflation unit supplies the gas to the first airbag unit and an air pressure in the space reaches and maintains a value, the signal sensing unit captures a continuous pulsation signal of a human body and transmits the pulsation signal to the processing unit; and
when the second airbag unit receives the gas supplied by the air inflation and deflation unit and continuously discharges the gas from the space, the pressure sensing unit continuously detects a change in air pressure in the space, and transmits a waveform signal generated by the change in air pressure to the processing unit.

8. The blood pressure measuring device capable of measuring meridians as claimed in claim 7, wherein the blood pressure measuring device further comprises a first valve unit and a second valve unit, the first valve unit is disposed at the downstream end of the air path to enable the air path to communicate externally or close from the outside, and the second valve unit is disposed on the air path and located between the first airbag unit and the second airbag unit.

9. The blood pressure measuring device capable of measuring meridians as claimed in claim 7, wherein the air path further comprises an air leakage part, and the air leakage part is located at the downstream end for the gas to be discharged from the space quantitatively.
